SEQUENCE LISTING (32 sequences)

SEQ ID NO: 1 - protein, 15 aa, Artificial sequence
  Amino Acid sequence of Cop 1-related peptide
AAAYAAAAAAKAAAA

SEQ ID NO: 2 - protein, 15 aa, Artificial sequence
  Amino Acid sequence of Cop 1-related peptide
AEKYAAAAAAKAAAA

SEQ ID NO: 3 - protein, 15 aa, Artificial sequence
  Amino Acid sequence of Cop 1-related peptide
AKEYAAAAAAKAAAA

SEQ ID NO: 4 - protein, 15 aa, Artificial sequence
  Amino Acid sequence of Cop 1-related peptide
AKKYAAAAAAKAAAA

SEQ ID NO: 5 - protein, 15 aa, Artificial sequence
  Amino Acid sequence of Cop 1-related peptide
AEAYAAAAAAKAAAA

SEQ ID NO: 6 - protein, 15 aa, Artificial sequence
  Amino Acid sequence of Cop 1-related peptide
KEAYAAAAAAKAAAA

SEQ ID NO: 7 - protein, 15 aa, Artificial sequence
  Amino Acid sequence of Cop 1-related peptide
AEEYAAAAAAKAAAA

SEQ ID NO: 8 - protein, 15 aa, Artificial sequence
  Amino Acid sequence of Cop 1-related peptide
AAEYAAAAAAKAAAA

SEQ ID NO: 9 - protein, 15 aa, Artificial sequence
  Amino Acid sequence of Cop 1-related peptide
EKAYAAAAAAKAAAA

SEQ ID NO: 10 - protein, 15 aa, Artificial sequence
  Amino Acid sequence of Cop 1-related peptide
AAKYEAAAAAKAAAA

SEQ ID NO: 11 - protein, 15 aa, Artificial sequence
  Amino Acid sequence of Cop 1-related peptide
AAKYAEAAAAKAAAA

SEQ ID NO: 12 - protein, 15 aa, Artificial sequence
  Amino Acid sequence of Cop 1-related peptide
EAAYAAAAAAKAAAA

SEQ ID NO: 13 - protein, 15 aa, Artificial sequence
  Amino Acid sequence of Cop 1-related peptide
EKKYAAAAAAKAAAA

SEQ ID NO: 14 - protein, 15 aa, Artificial sequence
  Amino Acid sequence of Cop 1-related peptide
EAKYAAAAAAKAAAA

SEQ ID NO: 15 - protein, 15 aa, Artificial sequence
  Amino Acid sequence of Cop 1-related peptide
AEKYAAAAAAAAAAA

SEQ ID NO: 16 - protein, 15 aa, Artificial sequence
  Amino Acid sequence of Cop 1-related peptide
AKEYAAAAAAAAAAA

SEQ ID NO: 17 - protein, 15 aa, Artificial sequence
  Amino Acid sequence of Cop 1-related peptide
AKKYEAAAAAAAAAA

SEQ ID NO: 18 - protein, 15 aa, Artificial sequence
  Amino Acid sequence of Cop 1-related peptide
AKKYAEAAAAAAAAA

SEQ ID NO: 19 - protein, 15 aa, Artificial sequence
  Amino Acid sequence of Cop 1-related peptide
AEAYKAAAAAAAAAA

SEQ ID NO: 20 - protein, 15 aa, Artificial sequence
  Amino Acid sequence of Cop 1-related peptide
KEAYAAAAAAAAAAA

SEQ ID NO: 21 - protein, 15 aa, Artificial sequence
  Amino Acid sequence of Cop 1-related peptide
AEEYKAAAAAAAAAA

SEQ ID NO: 22 - protein, 15 aa, Artificial sequence
  Amino Acid sequence of Cop 1-related peptide
AAEYKAAAAAAAAAA

SEQ ID NO: 23 - protein, 15 aa, Artificial sequence
  Amino Acid sequence of Cop 1-related peptide
EKAYAAAAAAAAAAA

SEQ ID NO: 24 - protein, 15 aa, Artificial sequence
  Amino Acid sequence of Cop 1-related peptide
AAKYEAAAAAAAAAA

SEQ ID NO: 25 - protein, 15 aa, Artificial sequence
  Amino Acid sequence of Cop 1-related peptide
AAKYAEAAAAAAAAA

SEQ ID NO: 26 - protein, 15 aa, Artificial sequence
  Amino Acid sequence of Cop 1-related peptide
EKKYAAAAAAAAAAA

SEQ ID NO: 27 - protein, 15 aa, Artificial sequence
  Amino Acid sequence of Cop 1-related peptide
EAKYAAAAAAAAAAA

SEQ ID NO: 28 - protein, 15 aa, Artificial sequence
  Amino Acid sequence of Cop 1-related peptide
AEYAKAAAAAAAAAA

SEQ ID NO: 29 - protein, 15 aa, Artificial sequence
  Amino Acid sequence of Cop 1-related peptide
AEKAYAAAAAAAAAA

SEQ ID NO: 30 - protein, 15 aa, Artificial sequence
  Amino Acid sequence of Cop 1-related peptide
EKYAAAAAAAAAAAA

SEQ ID NO: 31 - protein, 15 aa, Artificial sequence
  Amino Acid sequence of Cop 1-related peptide
AYKAEAAAAAAAAAA

SEQ ID NO: 32 - protein, 15 aa, Artificial sequence
  Amino Acid sequence of Cop 1-related peptide
AKYAEAAAAAAAAAA

(19) United States
(12) Patent Application Publication
(10) Pub. No.: US 2020/0397853 A1
(43) Pub. Date: Dec. 24, 2020

(54) TREATMENT OF A HEART DISEASE

(71) Applicant: Yeda Research and Development Co. Ltd., Rehovot (IL)

(72) Inventors: Eldad TZAHOR, Rehovot (IL); Ruth ARNON, Rehovot (IL); Rachel SARIG, Rehovot (IL); Rina AHARONI, Rehovot (IL); Kfir Baruch UMANSKY, Rehovot (IL)

(73) Assignee: Yeda Research and Development Co. Ltd., Rehovot (IL)

(21) Appl. No.: 17/015,187

(22) Filed: Sep. 9, 2020

Related U.S. Application Data
(63) Continuation of application No. PCT/IL2019/050273, filed on Mar. 12, 2019.
(60) Provisional application No. 62/641,434, filed on Mar. 12, 2018.

(51) Int. Cl. A61K 38/10; A61P 9/10

(52) U.S. Cl. CPC A61K 38/10; A61P 9/10

(57) ABSTRACT

A method of treating a heart disease in a subject in need thereof is provided. The method comprises administering to the subject a therapeutically effective amount of an agent selected from the group consisting of Copolymer 1, a Copolymer 1-related polypeptide and a Copolymer 1-related peptide, thereby treating the heart disease.

### RELATED APPLICATION/S

This application is a Continuation of PCT Patent Application No. PCT/IL2019/050273 having International filing date of Mar. 12, 2029, which claims the benefit of priority under 35 USC § 119(e) of U.S. Provisional Patent Application No. 62/641,434 filed on Mar. 12, 2018. The contents of the above applications are all incorporated by reference as if fully set forth herein in their entirety.

### SEQUENCE LISTING STATEMENT

The ASCII file, entitled 83886SequenceListing.txt, created on Sep. 9, 2019, comprising 8,882 bytes, submitted concurrently with the filing of this application is incorporated herein by reference.

### FIELD AND BACKGROUND OF THE INVENTION

The present invention, in some embodiments thereof, relates to the treatment of heart diseases.

Cardiovascular diseases (CVDs) have become the number one cause of morbidity and mortality worldwide^{1, 2}. An estimated 17.3 million people died from CVDs in 2015 and by 2030 more than 23 million people are expected to die annually. Thus, the overall market of products for myocardial repair/regeneration was estimated ˜$4 billion in the U.S. (similar to the European market). Among the various CVDs, Coronary Heart Disease (CHD), and particularly Myocardial Infarction (MI), i.e. heart attack, is the leading cause of death. In this injury, a coronary artery is occluded, in turn causing necrosis, inflammation and scarring of the heart³. The damage caused in this scenario can also lead to further deterioration of the heart, including dilated cardiomyopathy, chronic heart failure (CHF) and even ventricular wall rupture³. Mammalian cardiomyocytes (CMs) that make up the muscle of the heart, have a very limited regenerative capacity after injury. This has led to the long held dogma that endogenous CMs cannot be induced to repair a damaged heart. Cardiac regenerative medicine has focused on stem-cell therapy as a mean to replace the massive loss of CMs associated with CHD, however, the medical benefit of these studies in human patients is as yet uncertain. The only definitive treatment for heart failure is cardiac transplantation—an option that is limited by donor heart scarcity. The lack of a real therapeutic success among these intensive clinical efforts highlights the need for novel therapeutic approaches to cure heart diseases. Previous research in the field revealed the importance of the immune system in modulating the MI outcome (reviewed in⁴). The beneficial effects of T-regs and anti-inflammatory M2-macrophages in improving cardiac regeneration post MI were recently demonstrated^{5, 6}.

It is therefore compelling to look for an agent that can alter the immune response after MI from a pro-inflammatory to a pro-regenerative or protective response.

Glatiramer acetate (GA, Copaxone) is a synthetic random copolymer currently used as a first line treatment for multiple sclerosis (MS)⁷. The mechanism of action of GA has been investigated in the animal model of MS, experimental autoimmune encephalomyelitis (EAE) as well as in MS patients. These studies attributed the therapeutic activity of GA to its immunomodulatory effect at different levels of the innate and the adaptive immune response^{8, 9}. GA has also been shown to modulate the properties of dendritic cells and monocytes, so that they preferentially stimulate T-helper (Th)-2 like responses¹⁰. Indeed, GA is a potent inducer of Th2/3 cells that secrete high levels of anti-inflammatory cytokines¹¹⁻¹⁴. Additional studies demonstrated a reduction in the level of Th-17 cells in the CNS of EAE mice following GA treatment, with concomitant substantial elevation of T-regulatory cells (T-regs) 15, 16 Accumulated findings indicate that GA-treatment leads also to augmentation of neuroprotective and repair processes in the nervous system¹⁷, and amelioration of inflammatory bowel diseases (IBD)¹⁸⁻²⁰.

The beneficial effects of T-regs and anti-inflammatory M2-macrophages in improving cardiac regeneration post MI were recently demonstrated^{5, 6}.

As mentioned, Glatiramer acetate is used therapeutically in multiple sclerosis but is also known for adverse effects including elevated coronary artery disease (CAD) risk. The mechanisms underlying the cardiovascular side effects of the medication are unclear. (Brenne et al. PLoS One. 2017 Aug. 22; 12(8):e0182999).

Additional background art includes:

US Patent Application No. 20180051012, US Patent Application No. 20170233370 and International Patent Application Publication No. WO 2006/057003 teach the use of GA as a part of a combined specific treatment for various indications including MI. www(dot)sideeffects(dot)embl(dot)de/drugs/3081884/

### SUMMARY OF THE INVENTION

According to an aspect of some embodiments of the present invention there is provided a method of treating a heart disease in a subject in need thereof, the method comprising administering to the subject a therapeutically effective amount of an agent selected from the group consisting of Copolymer 1, a Copolymer 1-related polypeptide and a Copolymer 1-related peptide, thereby treating the heart disease.

According to an aspect of some embodiments of the present invention there is provided a therapeutically effective amount of an agent selected from the group consisting of Copolymer 1, a Copolymer 1-related polypeptide and a Copolymer 1-related peptide for use in the treatment of a heart disease in a subject in need thereof.

According to some embodiments of the invention, the heart disease is an ischemic heart disease.

According to some embodiments of the invention, the heart disease is a result of an acute adverse cardiac event.

According to some embodiments of the invention, the heart disease is a result of a chronic stress or disease of the heart.

According to some embodiments of the invention, the heart disease is selected from the group consisting of myocardial infarction (MI), chronic heart failure, angina pectoris, ischemic cardiomyopathy, heart failure, systemic hypertension, pulmonary hypertension, valve dysfunction, congestive heart failure and coronary artery disease.

According to some embodiments of the invention, the therapeutically effective amount results in a functional and/or anatomical repair of a damaged heart tissue.

According to some embodiments of the invention, the functional repair comprises an increase in cardiac output.

According to some embodiments of the invention, the increase in cardiac output comprises an increase in left ventricular ejection fraction (LVEF) of at least 5%.

According to some embodiments of the invention, the increase in cardiac output comprises an increase in left ventricular fractional shortening (LVFS) of at least 2%.

According to some embodiments of the invention, the therapeutically effective amount results in an anatomical repair of a damaged or diseased tissue comprising an increase in ventricular wall thickness and/or a decrease in scar tissue formation.

According to some embodiments of the invention, the agent is Copolymer 1.

According to some embodiments of the invention, the subject does not suffer from multiple sclerosis.

According to some embodiments of the invention, the therapeutically effective amount results in a modification in expression of cytokines at a cardiac lesion site.

According to some embodiments of the invention, the therapeutically effective amount results in Stat3 activation.

According to some embodiments of the invention, the therapeutically effective amount results neutrophils decrease and macrophages increase at a cardiac lesion site.

According to some embodiments of the invention, the method or agent as described herein further comprising administering to the subject a treatment for treating a heart disease other than the agent.

According to some embodiments of the invention, the method or agent as described herein with the proviso that the agent is not administered to the subject in combination with stem cells, a diacylglycerol acyltransferase 2 (DGAT2) inhibitor or a ferroptosis inhibitor.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D show various heart function parameters that were measured in mice post MI, comparing treated versus non-treated groups. FIG. 1A shows averages of Ejection Fraction (EF) measurements before MI (Time 0) and at 2 d, 14 d and 35 d post MI. FIG. 1B summarizes EF measurements at baseline (before MI) and 35 d after MI in PBS and GA treated groups (left). Right panel shows the percent reduction in EF at both groups, 35 d after MI. FIG. 1C summarizes fractional shortening (FS) measurements at baseline (before MI) and 35 d after MI in PBS and GA treated groups. FIG. 1D summarizes left ventricular left ventricular posterior wall (LVPW) measurements at baseline (before MI) and 35 d after MI in PBS and GA treated groups (left). Right panel shows the percent reduction in LVPW at both groups, 35 d after MI.

FIGS. 2A-C show a summary of various heart function parameters, measured as in FIGS. 1A-D, comparing treated versus non-treated mice, in a larger group. FIGS. 2A-B show echocardiographic measurements of ejection fraction (EF) and Fraction shortening (FS) parameters, respectively. Measurements are shown before surgery (baseline) and 35 days after myocardial infarction (MI) in hearts that were treated with either PBS or Copaxone (Cop) (left panels). The percent reduction in EF parameters is shown in the right panels. n=7 for PBS and n=13 for Cop. Data are presented as mean+/−s.e.m. Statistical significance was calculated using a two-tailed t-test, * P<0.05, ** P<0.01. FIG. 2C shows a heart section scar assessment following PBS or Cop treatment at 35 days after MI. Scarred area in all hearts was measured using ImageJ software and calculated as Masson-Trichrome stained area relative to total area size. Data are presented as mean+/−s.e.m. Statistical significance was calculated using a two-tailed t-test, * P<0.05.

FIGS. 3A-B describe scar area measurements in sectioned hearts of treated versus non-treated groups, 35 d post MI. Shown are representative sections of 3 mice in each group (FIG. 3A), followed with their individual EF measurements (FIG. 3B). FIG. 3A shows the representative sections, stained with Masson-Trichrome to observe the scar in PBS and GA treated mice. Right panel summarizes the averaged scar area in both groups. FIG. 3B shows the compatible EF measurements of the mice analyzed in FIG. 3A.

FIGS. 4A-B demonstrate a wide temporal therapeutic range of Copaxone. Echocardiographic measurements of ejection fraction (EF, FIG. 4A) and Fraction shortening (FS, FIG. 4B) parameters of animals treated with PBS or Cop at the day of injury, 24 or 48 hours post MI. n=9 for PBS, n=16 for Cop t0, n=7 for Cop 24 h, and n=5 for Cop 48 h. Data are presented as mean+/−s.e.m. Statistical significance was calculated using a two-tailed t-test, * P<0.05, ** P<0.01.

FIGS. 5A-D show the effect of Copaxone treatment after MI, which alters the immune cell population in the heart. FIGS. 5A-B) FACS analysis showing the percent of neutrophils in PBS and Cop treated hearts, at the indicated time points after MI. FIGS. 5C-D) FACS analysis showing the percent of macrophages in PBS and Cop treated hearts, at the indicated time points after MI.

FIGS. 6A-C show the effect of Copaxone treatment after MI, which alters cytokine levels in the heart. FIGS. 6A-C) Graphs summarizing ELISA analysis of PBS and Cop treated hearts, at the indicated time points post MI. n=4 for each group. Data are presented as mean+/−s.e.m. Statistical significance was calculated using a two-tailed t-test, * P<0.05, ** P<0.01.

FIG. 7 shows Western-blot analysis of PBS and Cop treated hearts, 4 days post MI. Cop treatment induced upregulation in the levels of pStat3.

### DESCRIPTION OF SPECIFIC EMBODIMENTS OF THE INVENTION

The present invention, in some embodiments thereof, relates to the treatment of heart diseases.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Whilst reducing embodiments of the invention to practice, the present inventors exploited the immunomodulatory and reparative outcomes of GA-treatment, to test its potential application for improving heart function after MI. Results presented in the Examples section which follows illustrate the beneficial effects of GA treatment in both improving cardiac function and reducing scar area in a mouse model of MI by LAD ligation. Furthermore, GA caused antiparallel change in neutrophil and macrophage populations 24 hr post MI, as evident from the decrease in neutrophil numbers and increase in macrophage number. In addition, GA caused an increase in the following cytokine secretion: IL-10, IL-6 and MCP-1, inducing activation of the transcription factor Stat3, shown to have beneficial effects repairing the heart after injury.

It is therefore contemplated to use GA as a therapeutic agent to improve heart function after acute MI as well in heart failure patients.

Thus, according to an aspect of the invention there is provided a method of treating a heart disease in a subject in need thereof, the method comprising administering to the subject a therapeutically effective amount of an agent selected from the group consisting of Copolymer 1, a Copolymer 1-related polypeptide and a Copolymer 1-related peptide, thereby treating the heart disease.

According to an aspect of the invention there is provided a therapeutically effective amount of an agent selected from the group consisting of Copolymer 1, a Copolymer 1-related polypeptide and a Copolymer 1-related peptide for use in the treatment of a heart disease in a subject in need thereof.

As used herein “heart disease”: refers to a class of diseases which onset or progression involves the heart.

The heart disease is a chronic heart disease.

The heart disease is an acute heart disease.

According to some embodiments of the invention, the heart disease is an ischemic heart disease.

According to a specific embodiment the ischemic heart disease is selected from the group consisting of acute myocardial infarction (AMI), myocardial infarction (MI), Chronic heart failure (CHF).

According to some embodiments of the invention, the heart disease is a result of an acute adverse cardiac event.

According to some embodiments of the invention, the heart disease is a result of a chronic stress or disease of the heart.

According to some embodiments of the invention, the heart disease is selected from the group consisting of myocardial infarction (MI), congenital heart disease, cardiac arrhythmias, heart failure, chronic heart failure, angina pectoris, ischemic cardiomyopathy, heart failure, systemic hypertension, pulmonary hypertension, valve dysfunction, congestive heart failure and coronary artery disease.

Ischemic Heart Disease

Heart ailments caused by narrowing of the coronary arteries and therefore a decreased blood supply to the heart.

Ischemic heart disease includes: angina, coronary artery disease, coronary heart disease, heart attack, and sudden death.

Hypertensive Heart Disease:

High blood pressure may overburden the heart and blood vessels and cause disease.

Hypertensive heart disease includes: aneurysm, and peripheral arterial disease.

Rheumatic Heart Disease

Rheumatic heart disease is caused by one or more attacks of rheumatic fever, which then do damage to the heart.

Rheumatic heart disease includes: valvular heart disease.

Inflammatory Heart Disease

Inflammation of the heart muscle (myocarditis), the membrane sac (pericarditis) which surrounds the heart, the inner lining of the heart (endocarditis) or the myocardium (heart muscle).

Inflammatory heart disease includes: cardiomyopathy, pericardial disease, and valvular heart disease.

Angina

Angina manifests as pain in the chest that results from reduced blood supply to the heart (ischemia). Angina is caused by atherosclerosis, that is the narrowing and/or blockage of the blood vessels that supply the heart.

The typical pain of angina is in the chest but it can often radiate to the left arm, shoulder or jaw.

Coronary Artery Disease

Coronary artery disease is caused by atherosclerosis, that is the narrowing and/or blockage of the blood vessels that supply the heart. It is one of the most common forms of heart disease and the leading cause of myocardial infarction (e.g. heart attacks) and angina.

Coronary Heart Disease

Coronary heart disease refers to the disease of the arteries to the heart and their resulting complications, such as angina and heart attacks.

Heart Attack

A heart attack (myocardial infarction) occurs when the heart's supply of blood is stopped.

Aneurysm

An aneurysm is a bulge or weakness in the wall of a blood vessel. Aneurysms can enlarge over time and may be life threatening if they rupture. They can occur because of high blood pressure or a weak spot in a blood vessel wall.

High Blood Pressure (Hypertension)

High blood pressure is the excessive force of blood pumping through blood vessels. High blood pressure causes many types of cardiovascular disease, such as heart failure, and renal disease.

Rheumatic Heart Disease

Rheumatic heart disease is damage caused to the heart's valves by rheumatic fever, which is caused by streptococcal bacteria.

Valvular Heart Disease

The heart's valves keep blood flowing through the heart in the right direction. But a variety of conditions can lead to valvular damage. Valves may narrow (stenosis), leak (regurgitation or insufficiency) or not close properly (prolapse). Valvular disease can be congenital, or the valves may be damaged by such conditions as rheumatic fever, infections connective tissue disorders, and certain medications or radiation treatments for cancer.

Cardiomyopathy

Cardiomyopathy refers to diseases of the heart muscle. Some types of cardiomyopathy are genetic, while others occur because of infection or other reasons that are less well understood. One of the most common types of cardiomyopathy is idiopathic dilated cardiomyopathy, where the heart is enlarged. Other types include ischemic, loss of heart muscle; dilated, heart enlarged; hypertrophic, heart muscle is thickened.

Valvular Heart Disease

The heart's valves keep blood flowing through the heart in the right direction. But a variety of conditions can lead to valvular damage. Valves may narrow (stenosis), leak (regurgitation or insufficiency) or not close properly (prolapse). Valvular disease may be congenital or the valves may be damaged by such conditions as rheumatic fever, infections connective tissue disorders, and certain medications or radiation treatments for cancer.

Heart Failure

Heart failure is a chronic condition that happens when the heart's muscle becomes too damaged to adequately pump the blood around the body.

Cardiac Arrhythmias

An arrhythmia is a problem with the rate or rhythm of the heartbeat, where the heart beats irregularly, too fast or too slow. Common types of arrhythmias include atrial fibrillation (the heart contracts in an irregular way at a high rate), bradycardia (when the heart beats irregularly or too slow), and supraventricular tachycardia (when the heart beats irregularly or too fast).

As used herein “subject in need thereof” refers to a subject diagnosed with a heart disease.

The term “treating” refers to inhibiting, preventing or arresting the development of a pathology (i.e., heart disease, e.g., ischemic heart disease) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings at any age that is diagnosed with the pathology (i.e., heart disease). According to a specific embodiment, the subject does not have multiple sclerosis.

According to a specific embodiment the subject is an adult i.e., above 21 years of age.

According to a specific embodiment the subject is an adolescent i.e., 12-21 years of age.

According to a specific embodiment the subject is a child i.e., 2-12 years of age.

According to a specific embodiment the subject is an infant i.e., 1 month to 2 years of age.

According to a specific embodiment the subject is a newborn i.e., birth to 1 month of age.

According to a specific embodiment the subject is a male.

According to a specific embodiment the subject is a female.

As used herein in the application, the terms “Cop 1”, “Copolymer 1”, “glatiramer acetate” and “GA” are used interchangeably.

For the purpose of the present invention, “Copolymer 1 or a Copolymer 1-related peptide or polypeptide” is intended to include any peptide or polypeptide, including a random copolymer that cross-reacts functionally with MBP and is able to compete with MBP on the MHC class II in the antigen presentation.

The composition for use in the invention may comprise as active agent a Cop 1 or a Cop 1-related peptide or polypeptide represented by a random copolymer consisting of a suitable ratio of a positively charged amino acid such as lysine or arginine, in combination with a negatively charged amino acid (e.g., in a lesser quantity) such as glutamic acid or aspartic acid, optionally in combination with a non-charged neutral amino acid such as alanine or glycine, serving as a filler, and optionally with an amino acid adapted to confer on the copolymer immunogenic properties, such as an aromatic amino acid like tyrosine or tryptophan. Such compositions may include any of those copolymers disclosed in WO 00/05250, the entire contents of which are herewith incorporated herein by reference.

According to some embodiments, the composition for use in the present invention comprises at least one copolymer selected from the group consisting of random copolymers comprising one amino acid selected from each of at least three of the following groups: (a) lysine and arginine; (b) glutamic acid and aspartic acid; (c) alanine and glycine; and (d) tyrosine and tryptophan.

According to some embodiments, the copolymers for use in the present invention can be composed of L- or D-amino acids or mixtures thereof. As is known by those of skill in the art, L-amino acids occur in most natural proteins. However, D-amino acids are commercially available and can be substituted for some or all of the amino acids used to make the copolymers used in the present invention. According to some embodiments, the present invention contemplates the use of copolymers containing both D- and L-amino acids, as well as copolymers consisting essentially of either L- or D-amino acids.

In one embodiment of the invention, the copolymer contains four different amino acids, each from a different one of the groups (a) to (d).

According to some embodiments, the composition comprises Copolymer 1, a mixture of random polypeptides consisting essentially of the amino acids L-glutamic acid (E), L-alanine (A), L-tyrosine (Y) and L-lysine (K) in an approximate ratio of 1.5:4.8:1:3.6, having a net overall positive electrical charge and of a molecular weight from about 2 KDa to about 40 KDa.

According to some embodiments, the Cop 1 has average molecular weight of about 2 KDa to about 20 KDa, about 4 KDa, 7 KDa to about 13 K Da, still about 4 KDa to about 8.6 KDa, of about 5 KDa to 9 KDa, or of about 6.25 KDa to 8.4 KDa. In another embodiment, the Cop 1 has average molecular weight of about 13 KDa to about 20 KDa, about 13.5 KDa to about 18 KDa, with an average of about 15 KDa to about 16 KD, e.g., of 16 kDa. Other average molecular weights for Cop 1, lower than 40 KDa, are also encompassed by the present invention. Copolymer 1 of said molecular weight ranges can be prepared by methods known in the art, for example by the processes described in U.S. Pat. No. 5,800,808, the entire contents of which are hereby incorporated by reference in the entirety. The Copolymer 1 may be a polypeptide comprising from about 15 to about 100, e.g., from about 40 to about 80, amino acids in length.

In one embodiment of the invention, the agent is Cop 1 in the form of its acetate salt known under the generic name glatiramer acetate or its trade name Copaxone® (a trademark of Teva Pharmaceutical Industries Ltd., Petach Tikva, Israel).

The activity of Copolymer 1 for the composition disclosed herein is expected to remain if one or more of the following substitutions is made: aspartic acid for glutamic acid, glycine for alanine, arginine for lysine, and tryptophan for tyrosine.

In another embodiment of the invention, the Cop 1-related peptide or polypeptide is a copolymer of three different amino acids each from a different one of three groups of the groups (a) to (d). These copolymers are herein referred to as terpolymers.

In one embodiment, the Cop 1-related peptide or polypeptide is a terpolymer containing tyrosine, alanine, and lysine, hereinafter designated YAK, in which the average molar fraction of the amino acids can vary: tyrosine can be present in a mole fraction of about 0.05-0.250; alanine in a mole fraction of about 0.3-0.6; and lysine in a mole fraction of about 0.1-0.5. According to some embodiments, the molar ratios of tyrosine, alanine and lysine are about 0.10:0.54:0.35, respectively. It is possible to substitute arginine for lysine, glycine for alanine, and/or tryptophan for tyrosine.

In another embodiment, the Cop 1-related peptide or polypeptide is a terpolymer containing tyrosine, glutamic acid, and lysine, hereinafter designated YEK, in which the average molar fraction of the amino acids can vary: glutamic acid can be present in a mole fraction of about 0.005-0.300, tyrosine can be present in a mole fraction of about 0.005-0.250, and lysine can be present in a mole fraction of about 0.3-0.7. According to some embodiments, the molar ratios of glutamic acid, tyrosine, and lysine are about 0.26:0.16:0.58, respectively. It is possible to substitute aspartic acid for glutamic acid, arginine for lysine, and/or tryptophan for tyrosine.

According to some embodiments, the Cop 1-related peptide or polypeptide is a terpolymer containing lysine, glutamic acid, and alanine, hereinafter designated KEA, in which the average molar fraction of the amino acids can vary: glutamic acid can be present in a mole fraction of about 0.005-0.300, alanine in a mole fraction of about 0.005-0.600, and lysine can be present in a mole fraction of about 0.2-0.7. According to some embodiments, the molar ratios of glutamic acid, alanine and lysine are about 0.15:0.48:0.36, respectively. It is possible to substitute aspartic acid for glutamic acid, glycine for alanine, and/or arginine for lysine.

According to some embodiments, the Cop 1-related peptide or polypeptide is a terpolymer containing tyrosine, glutamic acid, and alanine, hereinafter designated YEA, in which the average molar fraction of the amino acids can vary: tyrosine can be present in a mole fraction of about 0.005-0.250, glutamic acid in a mole fraction of about 0.005-0.300, and alanine in a mole fraction of about 0.005-0.800. According to some embodiments, the molar ratios of glutamic acid, alanine, and tyrosine are about 0.21:0.65:0.14, respectively. It is possible to substitute tryptophan for tyrosine, aspartic acid for glutamic acid, and/or glycine for alanine.

The average molecular weight of the terpolymers YAK, YEK, KEA and YEA can vary between about 2 KDa to 40 KDa, e.g., between about 3 KDa to 35 KDa, e.g., between about 5 KDa to 25 KDa.

Copolymer 1 and related peptides and polypeptides may be prepared by methods known in the art, for example, under condensation conditions using the desired molar ratio of amino acids in solution, or by solid phase synthetic procedures. Condensation conditions include the proper temperature, pH, and solvent conditions for condensing the carboxyl group of one amino acid with the amino group of another amino acid to form a peptide bond. Condensing agents, for example dicyclohexylcarbodiimide, can be used to facilitate the formation of the peptide bond. Blocking groups can be used to protect functional groups, such as the side chain moieties and some of the amino or carboxyl groups against undesired side reactions.

For example, the copolymers can be prepared by the process disclosed in U.S. Pat. No. 3,849,550, wherein the N-carboxyanhydrides of tyrosine, alanine, γ-benzyl glutamate and N ε-trifluoroacetyl-lysine are polymerized at ambient temperatures (20° C.-26° C.) in anhydrous dioxane with diethylamine as an initiator. The γ-carboxyl group of the glutamic acid can be deblocked by hydrogen bromide in glacial acetic acid. The trifluoroacetyl groups are removed from lysine by 1M piperidine. One of skill in the art readily understands that the process can be adjusted to make peptides and polypeptides containing the desired amino acids, that is, three of the four amino acids in Copolymer 1, by selectively eliminating the reactions that relate to any one of glutamic acid, alanine, tyrosine, or lysine.

The molecular weight of the copolymers can be adjusted during polypeptide synthesis or after the copolymers have been made. To adjust the molecular weight during polypeptide synthesis, the synthetic conditions or the amounts of amino acids are adjusted so that synthesis stops when the polypeptide reaches the approximate length that is desired. After synthesis, polypeptides with the desired molecular weight can be obtained by any available size selection procedure, such as chromatography of the polypeptides on a molecular weight sizing column or gel, and collection of the molecular weight ranges desired. The copolymers can also be partially hydrolyzed to remove high molecular weight species, for example, by acid or enzymatic hydrolysis, and then purified to remove the acid or enzymes.

In one embodiment, the copolymers with a desired molecular weight may be prepared by a process, which includes reacting a protected polypeptide with hydrobromic acid to form a trifluoroacetyl-polypeptide having the desired molecular weight profile. The reaction is performed for a time and at a temperature that is predetermined by one or more test reactions. During the test reaction, the time and temperature are varied and the molecular weight range of a given batch of test polypeptides is determined. The test conditions that provide the optimal molecular weight range for that batch of polypeptides are used for the batch. Thus, a trifluoroacetyl-polypeptide having the desired molecular weight profile can be produced by a process, which includes reacting the protected polypeptide with hydrobromic acid for a time and at a temperature predetermined by test reaction. The trifluoroacetyl-polypeptide with the desired molecular weight profile is then further treated with an aqueous piperidine solution to form a low toxicity polypeptide having the desired molecular weight.

According to some embodiments, a test sample of protected polypeptide from a given batch is reacted with hydrobromic acid for about 10-50 hours at a temperature of about 20-28° C. The best conditions for that batch are determined by running several test reactions. For example, in one embodiment, the protected polypeptide is reacted with hydrobromic acid for about 17 hours at a temperature of about 26° C.

As binding motifs of Cop 1 to MS-associated HLA-DR molecules are known (Fridkis-Hareli et al, 1999), polypeptides derived from Cop 1 having a defined sequence can readily be prepared and tested for binding to the peptide binding groove of the HLA-DR molecules as described in the Fridkis-Hareli et al (1999) publication. Examples of such peptides are those disclosed in WO 00/05249 and WO 00/05250, the entire contents of which are hereby incorporated herein by reference, and include the peptides of SEQ ID NOs. 1-32 hereinbelow.

| SEQ ID NO. | Peptide Sequence |
|---|---|
| 1 | AAAYAAAAAAKAAAA |
| 2 | AEKYAAAAAAKAAAA |
| 3 | AKEYAAAAAAKAAAA |
| 4 | AKKYAAAAAAKAAAA |
| 5 | AEAYAAAAAAKAAAA |
| 6 | KEAYAAAAAAKAAAA |
| 7 | AEEYAAAAAAKAAAA |
| 8 | AAEYAAAAAAKAAAA |
| 9 | EKAYAAAAAAKAAAA |
| 10 | AAKYEAAAAAKAAAA |
| 11 | AAKYAEAAAAKAAAA |
| 12 | EAAYAAAAAAKAAAA |
| 13 | EKKYAAAAAAKAAAA |
| 14 | EAKYAAAAAAKAAAA |
| 15 | AEKYAAAAAAAAAAA |
| 16 | AKEYAAAAAAAAAAA |
| 17 | AKKYEAAAAAAAAAA |
| 18 | AKKYAEAAAAAAAAA |
| 19 | AEAYKAAAAAAAAAA |
| 20 | KEAYAAAAAAAAAAA |
| 21 | AEEYKAAAAAAAAAA |
| 22 | AAEYKAAAAAAAAAA |
| 23 | EKAYAAAAAAAAAAA |
| 24 | AAKYEAAAAAAAAAA |
| 25 | AAKYAEAAAAAAAAA |
| 26 | EKKYAAAAAAAAAAA |
| 27 | EAKYAAAAAAAAAAA |
| 28 | AEYAKAAAAAAAAAA |
| 29 | AEKAYAAAAAAAAAA |
| 30 | EKYAAAAAAAAAAAA |
| 31 | AYKAEAAAAAAAAAA |
| 32 | AKYAEAAAAAAAAAA |

Such peptides and other similar peptides derived from Cop 1 would be expected to have similar activity as Cop 1. Such peptides, and other similar peptides, are also considered to be within the definition of Cop 1-related peptides or polypeptides and their use is considered to be part of the present invention.

The definition of “Cop 1-related peptide or polypeptide” according to the invention is meant to encompass other synthetic amino acid copolymers such as the random four-amino acid copolymers described by Fridkis-Hareli et al., 2002 (as candidates for treatment of multiple sclerosis), namely copolymers (14-, 35- and 50-mers) containing the amino acids phenylalanine, glutamic acid, alanine and lysine (poly FEAK), or tyrosine, phenylalanine, alanine and lysine (poly YFAK), and any other similar copolymer to be discovered that can be considered a universal antigen similar to Cop 1.

According to a specific embodiment, the therapeutically effective amount results in a functional and/or anatomical repair of a damaged heart tissue.

According to a specific embodiment, the functional repair comprises an increase in cardiac output.

According to a specific embodiment, the increase in cardiac output comprises an increase in left ventricular ejection fraction (LVEF) of at least 2%, 5%, 7%, 10%, 15% 20%, e.g., 17.5%.

According to a specific embodiment, the increase in cardiac output comprises an increase in fractional shortening LV(FS) of at least 2%, 5%, 7%, 10%, 15% 20%, e.g., 3.6%.

According to a specific embodiment, the therapeutically effective amount results in an anatomical repair of a damaged or diseased tissue comprising an increase in ventricular wall thickness and/or a decrease in scar tissue formation.

According to a specific embodiment, the therapeutically effective amount results in a modulation (increase or decrease) in expression of cytokines at a cardiac lesion site.

According to a specific embodiment, the therapeutically effective amount results in increased expression IL-6, IL-10, IL-4 and MCP-1 at a cardiac lesion site, at least up to 4 days following the medical event leading to the heart disease.

According to a specific embodiment, the therapeutically effective amount results in an inhibition of expression IL-la, IL-6, IL-3, and GM-CSF at a cardiac lesion site, at least up to 14 days following the medical event leading to the heart disease.

According to a specific embodiment, the therapeutically effective amount results in Stat3 activation.

According to a specific embodiment, the therapeutically effective amount results neutrophils decrease and macrophages increase at a cardiac lesion site.

Methods of assessing gene expression, and cell characterization are well known in the art. Some are listed in the Examples section which follows but are not meant to be binding.

The dosage of Cop 1 to be administered will be determined by the physician according to the age of the patient and stage of the disease and may be chosen from a range of 1-80 mg, e.g., 20 mg, although any other suitable dosage is encompassed by the invention. Cop 1 may be administered daily e.g., for 7-14 days. In another embodiment, the administration may be made according to a regimen suitable for immunization, for example, at least once a month or at least once every 2 or 3 months, or less frequently, but any other suitable interval between the immunizations is envisaged by the invention according to the condition of the patient. Pharmaceutical compositions for use in accordance with the present invention may be formulated in conventional manner using one or more physiologically acceptable carriers or excipients. The carrier(s) must be “acceptable” in the sense of being compatible with the other ingredients of the composition and not deleterious to the recipient thereof.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical and intradermal routes, with or without adjuvant. Administration can be systemic or local (e.g., intracoronary administration using a catheter).

The amount of a composition to be administered will, of course, be dependent on the subject being treated, the severity of the affliction, the manner of administration, the judgment of the prescribing physician, etc.

According to a specific embodiment the therapeutically effective amount comprises a single administration.

According to a specific embodiment, the treatment is an acute treatment (e.g., dependent on the indication).

Non-limiting examples relate to 20 mg/day, by daily s.c. injections, or a double dose, every 48 h, for up to 14 days (e.g., starting at the day of the cardiac event e.g., MI, and up to 48 h post the event, e.g., MI).

Alternatively or additionally administration, GA may be administered such in the above contemplated doses for a period of several week, e.g., at least 2 weeks, at least 4 weeks, at least 5 weeks, at least 6 weeks, at least 7 weeks, at least 8 weeks, at least 3 months, at least 6 months, at least 12 months or more.

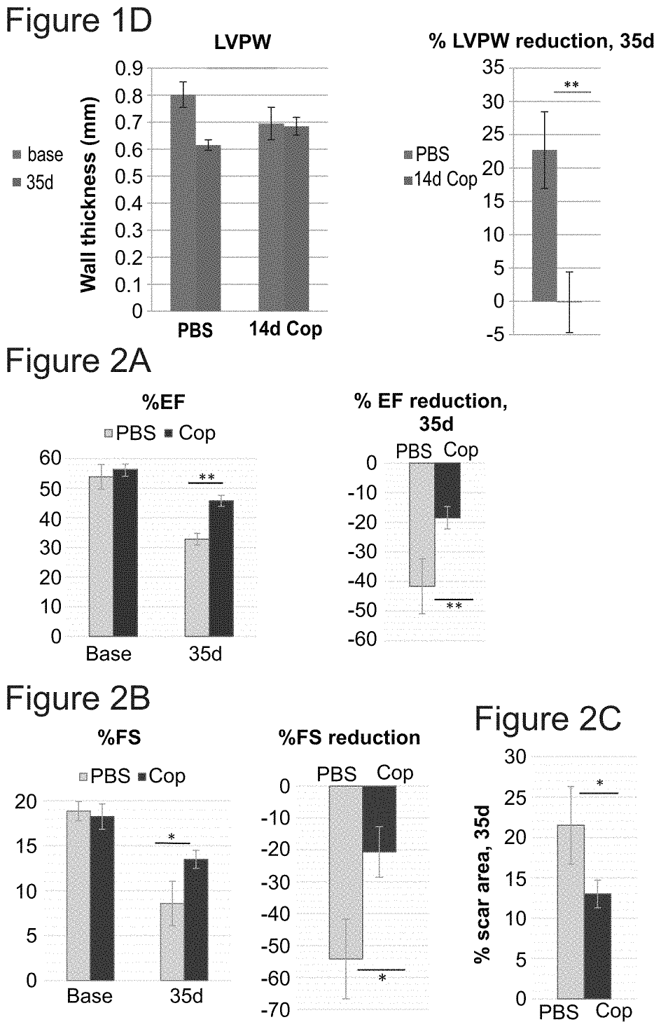

FIGS. 2A-C support a role for GA as a potent pro-regenerative agent after MI. It is suggested that longer treatment may yield improved heart function and reduce scar area.

Thus according to a specific embodiment, a chronic regimen is envisaged.

In a chronic administration regimen, GA may be administered such in the above contemplated doses for a period of several week, e.g., at least 2 weeks, at least 4 weeks, at least 5 weeks, at least 6 weeks, at least 7 weeks, at least 8 weeks, at least 3 months, at least 6 months, at least 12 months or more.

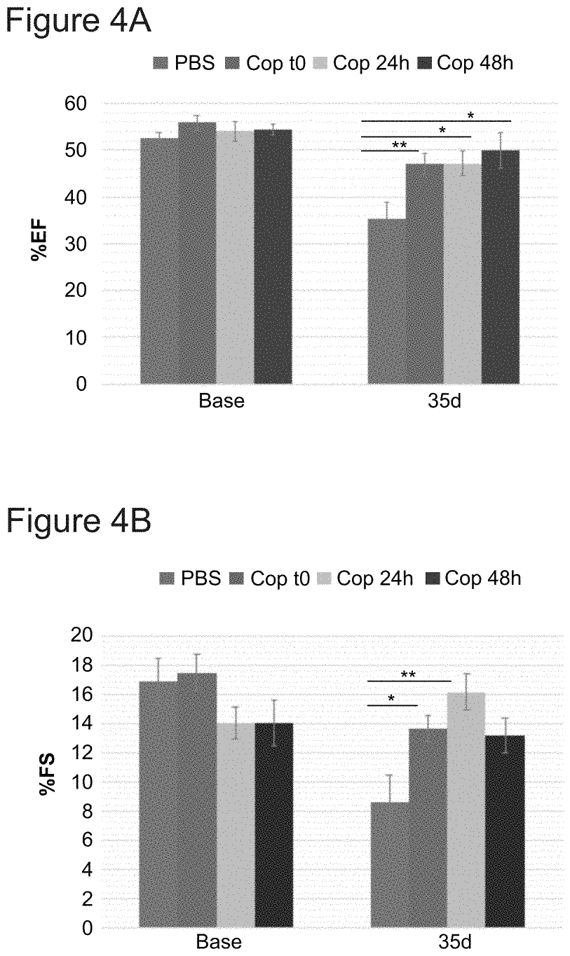

As shown in FIGS. 4A-B, treatment at both 24 and 48 hours delay resulted with improved cardiac function, reaching similar levels to those observed after treatment at the day of injury. This result suggests a wide temporal therapeutic range of GA, rendering it more attractive for patients that suffered a delay in reaching medical attention.

Thus, according to a specific embodiment, the therapeutic window, i.e., time of administration is 0-48 h following the event e.g., outbreak of the disease e.g., MI.

According to a specific embodiment, treatment with the agent is accompanied by other Gold standard treatments contemplated for heart diseases. The selection of the specific treatment depends on the specific type of the heart disease.

Exemplary treatments include, but are not limited to, Ace inhibitors, anticoagulation drugs, Beta blockers, Statins.

A more detailed list can be found at:

Specific examples include, but are not limited to:

Angiotensin-Converting Enzyme (ACE) Inhibitors

Commonly prescribed include:

Captopril (Capoten)

Enalapril (Vasotec)

Fosinopril (Monopril)

Lisinopril (Prinivil, Zestril)

Perindopril (Aceon)

Quinapril (Accupril)

Ramipril (Altace)

Trandolapril (Mavik)

Angiotensin II Receptor Blockers (or Inhibitors)

(Also known as ARBs or Angiotensin-2 Receptor Antagonists)

Commonly prescribed include:

Candesartan (Atacand)

Losartan (Cozaar)

Valsartan (Diovan)

Angiotensin-Receptor Neprilysin Inhibitors (ARNIs)

ARNIs are a new drug combination of a neprilysin inhibitor and an ARB.

Sacubitril/valsartan

I^{f} Channel Blocker (or Inhibitor)

This drug class reduces the heart rate, similar to another class of drugs called beta blockers.

Ivabradine (Corlanor)

Beta Blockers

(Also known as Beta-Adrenergic Blocking Agents)

Commonly prescribed include:

Bisoprolol (Zebeta)

Metoprolol succinate (Toprol XL)

Carvedilol (Coreg)

Carvedilol CR (Coreg CR) Toprol XL

Aldosterone Antagonists

Commonly prescribed include:

Spironolactone (Aldactone)

Eplerenone (Inspra)

Hydralazine and Isosorbide Dinitrate (Specifically Benefits African Americans with Heart Failure)

Commonly prescribed:

Hydralazine and isosorbide dinitrate (combination drug)—(Bidil)

Diuretics

(Also known as Water Pills)

Commonly prescribed include:

Furosemide (Lasix)

Bumetanide (Bumex)

Torsemide (Demadex)

Chlorothiazide (Diuril)

Amiloride (Midamor Chlorthalidone (Hygroton)

Hydrochlorothiazide or HCTZ (Esidrix, Hydrodiuril)

Indapamide (Lozol)

Metolazone (Zaroxolyn)

Triamterene (Dyrenium)

According to a specific embodiment, the agent is not administered to the subject in combination with stem cells, a diacylglycerol acyltransferase 2 (DGAT2) inhibitor or a ferroptosis inhibitor (i.e., as described in 20170233370 e.g., formulae I-IV, which is hereby incorporated by reference in its entirety).

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Methodology

Myocardial Infarction

Myocardial infarction in adult mice was performed as follows: mice were sedated with isoflurane (Abbott Laboratories) and following tracheal intubation, were artificially ventilated. Lateral thoracotomy at the third intercostal space was performed by blunt dissection of the intercostal muscles following skin incision. Following ligation of the left anterior descending coronary artery, 2 mg/animal/day of

GA (batch #164F0105; 24992916) or PBS were injected according to the experimental protocol (described in the Results). Following treatment, thoracic wall incisions were sutured with 6.0 non-absorbable silk sutures, and the skin wound closed using a skin adhesive. Mice were then warmed for several minutes until recovery.

Echocardiography

Heart function was evaluated by transthoracic echocardiography performed on sedated mice (isoflurane, Abbott Laboratories) using a Vevo 3100 VisualSonics device.

Histology

Mouse heart tissues were fixed in 4% paraformaldehyde (PFA) and sectioned. For analysis of cardiac regeneration, following myocardial infarction procedure, paraffin sections were cut through the entire ventricle from apex to base into serial sections with intervals of 0.3 mm. Paraffin sections were cut frontally to include base to apex in each section. Haematoxylin-eosin (H&E) and Masson's trichrome staining were performed according to standard procedures and used to for detection of fibrosis. Scar size was quantified in the section containing the papillary muscle region using ImageJ software based on Masson's trichrome staining. Scar size was calculated as scar size relative to total section size.

Flow Cytometry

Single cell suspensions of hearts harvested at different time point post MI were generated immediately before analysis by flow cytometry, as previously described²¹. Briefly, single hearts were minced and digested in collagenase II/Dispase solution in PBS with 1 mM CaCl₂ (2 mg/ml collagenase II, 1.2 U/ml Dispase) for 3 rounds of 15 minutes at 37° C., followed by mechanical separation by pippetation. Digested samples were passed through a 40-μm filter, washed, and suspended in FACS buffer (PBS 0.5% BSA, 2 mM EDTA) for staining. Samples were stained with antibodies from Miltenyi biotec:CD45-Viogreen, CD11b-PE, Ly6G-Vioblue, Ly6C-FITC, F4/80-APC, CCR2-APC-Vio770, MHCII-PE-Vio770. 7-AAD was used as a viability marker. Staining and sample analysis were all performed by standard procedure on a LSR-II FACS machine.

Cytokine ELISA

For native protein extraction, hearts were minced using a mortar and pestle under liquid nitrogen, and the resulting powder was further homogenized using rotor—stator homogenizer with a native extraction buffer (0.5% triton in PBS with protease inhibitor cocktail). Resulting extracts were equilibrated for protein concentration, and subjected to mouse cytokine 16-plex ELISA (#110949MS, Quansys Biosciences, USA) according to manufacturer instructions.

Western Blot Analysis

Western blotting was performed with the SDS-PAGE Electrophoresis System. Sample extracts were prepared and transferred to PVDF membranes. The following primary antibodies were used: anti-Gapdh (Sigma, PLA0125, 1:3000), anti-pStat3 (1:1000) Horseradish peroxidase anti-mouse, anti-rabbit or anti-goat (Sigma-Aldrich, 1:2000) were used as a secondary antibody.

### Example 1

### GA (Cop) Treatment Results in Improved Cardiac Function after MI

Experimental Outline

Mice were divided to the following experimental groups:

1. Sham—opening the chest cavity without MI, serves as untreated control. N=4.

2. PBS—performing MI followed by PBS treatment for up to 14 days. N=5.

3. Short GA treatment (i.p. only)—performing MI followed by GA (2 mg/animal/day) given i.p. from day 0 up to day 7. N=5.

4. Long GA treatment—performing MI followed by GA (2 mg/animal/day) given i.p. from day 0 up to day 14 or until first animal dies. N=5.

5. Long GA treatment (i.m.)—performing MI followed by GA (2 mg/animal/day) given i.m at day 0 and then given i.p. up to day 14 or until first animal dies. N=5.

Echo measurements were performed at baseline (day 0), day 2, day 14 and day 35 post MI. Histology analysis was performed post mortem.

Results

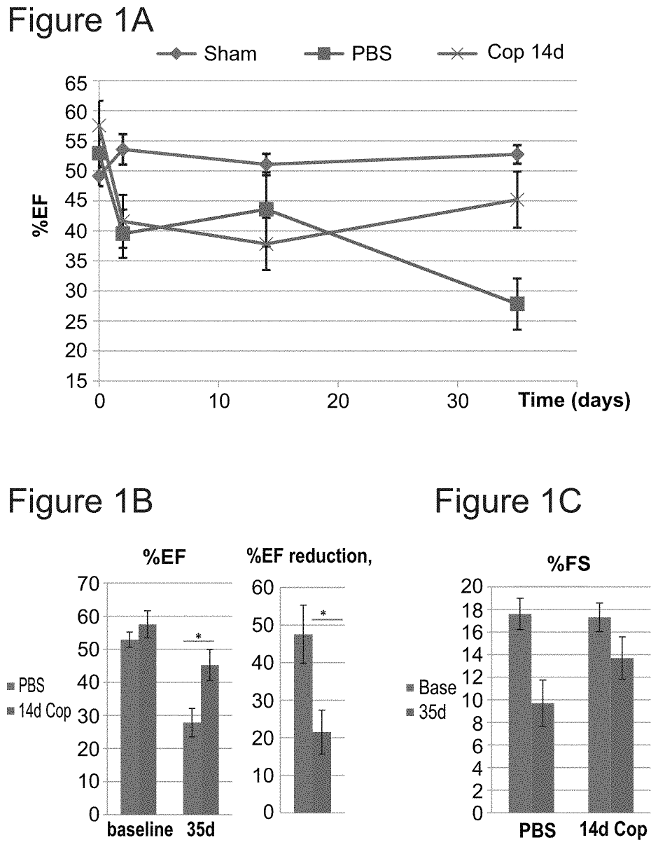

The group of treated mice that exhibited the most pronounced beneficial effects was group #4 (i.p. injections for 14 d). Therefore, all the presented data focus on the comparison between groups #1, #2 and #4 (Sham, PBS and GA treated for 14 d, respectively). FIGS. 1A-C show various heart function parameters that were measured in mice post MI, comparing treated versus non-treated groups. FIG. 1A shows averages of Ejection Fraction (EF) measurements before MI (Time 0) and at 2 d, 14 d and 35 d post MI. Sham and PBS groups included 4 mice each, and the group of mice that were treated with GA for 14 days consisted of 5 mice. A clear beneficial effect was observed 35 d post injury, in the GA treated group. FIG. 1B summarizes EF measurements at baseline (before MI) and 35 d after MI in PBS and GA treated groups (left). Right panel shows the percent reduction in EF at both groups, 35 d after MI. A statistically significant difference is observed between the two groups. FIG. 1C summarizes FS measurements at baseline (before MI) and 35 d after MI in PBS and GA treated groups. A smaller reduction is observed in GA treated group. FIG. 1D summarizes LVPW measurements at baseline (before MI) and 35 d after MI in PBS and GA treated groups (left). Right panel shows the percent reduction in LVPW at both groups, 35 d after MI. A statistically significant difference is observed between the two groups.

### Example 2

### Long GA Treatment after MI

To substantiate the MI results, the following mice groups were used:

Sham—opening the chest cavity without MI, served as an untreated control. N=5.

PBS—performing MI followed by PBS treatment (i.p.) for up to 21 days. N=10.

Short GA treatment—performing MI followed by GA (2 mg/animal/day) administered i.p. from day 0 up to day 14. N=10.

Long GA treatment—performing MI followed by GA (2 mg/animal/day) administered i.p. from day 0 up to day 21 or until first animal dies. N=10.

Echo measurements are performed at baseline (day 0), day 2, day 14 and day 35 post MI. Histology analysis is performed post mortem.

FIGS. 2A-C summarize data obtained from all the animals that were treated according to this protocol revealing a significant pro-regenerative effect in the GA treated group: both cardiac function (EF and FS, FIGS. 2A, B) and cardiac scarring (FIG. 2C) were improved significantly. This data underscores GA as a potent pro-regenerative agent after MI. It is suggested that longer treatment may yield improved heart function and reduce scar area. To determine GA mode of action, short-term experiments are performed in which GA and PBS treated hearts are collected at day 2, 4, 7 and 14 post MI, and are subjected to immunohistochemistry analysis to follow the immune cell composition, as well as the potential effect of GA on the proliferation capacity of CMs.

### Example 3

### GA Treatment Results in Reduced Scar Area after MI

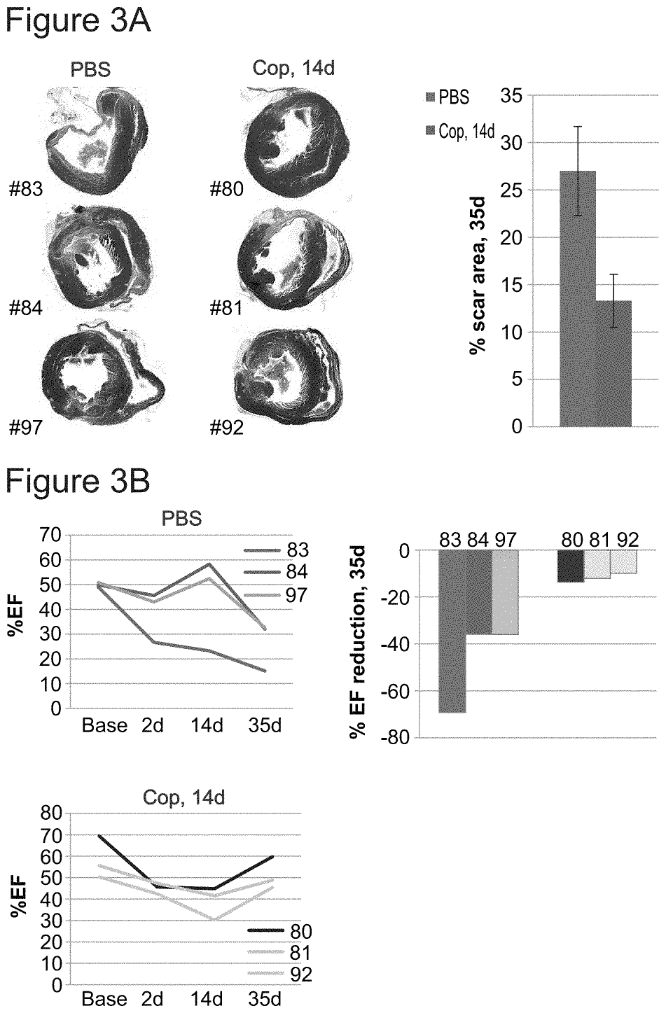

FIGS. 3A-B describe scar area measurements in sectioned hearts of treated versus non-treated groups, 35 d post MI. Shown are representative sections of 3 mice in each group (FIG. 3A), followed with their individual EF measurements (FIG. 3B). FIG. 3A shows the representative sections, stained with Masson-Trichrome to observe the scar in PBS and GA treated mice. Right panel summarizes the averaged scar area in both groups. FIG. 3B shows comparable EF measurements of the mice analyzed in FIG. 3A. All 3 mice that were treated exhibited improved EF and a smaller scar area.

### Example 4

### A Wide Temporal Therapeutic Range of GA

In the clinic, MI patients currently reach medical care in a matter of several hours. To find out the relevance and efficacy of delayed administration of GA, the present inventors created MI by LAD ligation and treated animals at the day of injury, 24 or 48 hours post MI. Interestingly, treatment at both 24 and 48 hours delay resulted with improved cardiac function (FIGS. 4A-B), reaching similar levels to those observed after treatment at the day of injury. This result suggests a wide temporal therapeutic range of GA, making it more appealing as it can be used even on patients that suffered a delay in reaching medical attention.

### Example 5

### GA Affects Immune Cell Population after MI

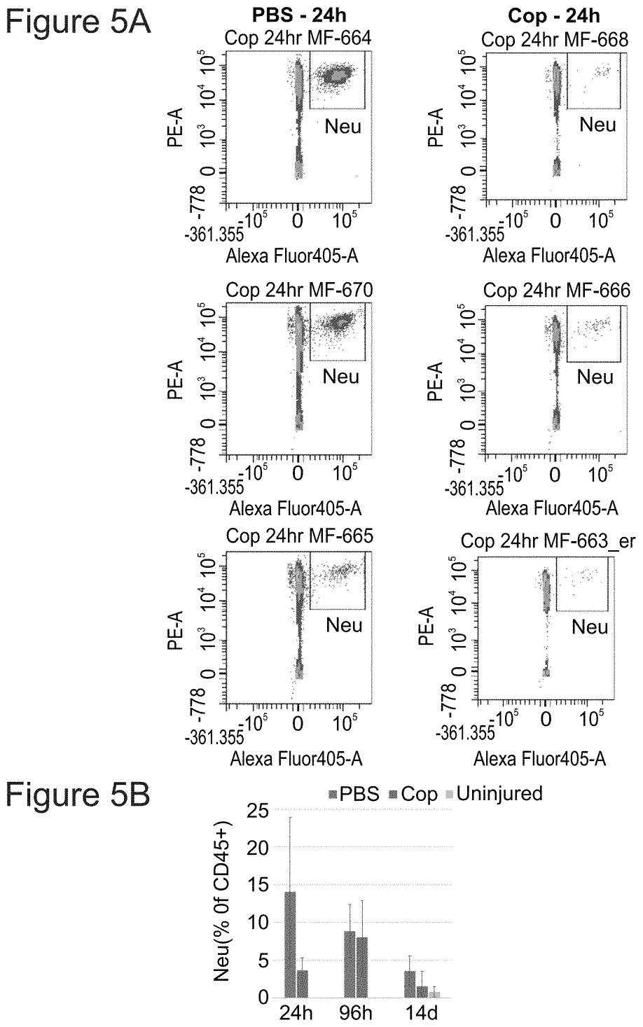

GA has an established influence on the immune response, especially the T-helper 2 (Th2) lymphocyte population²²⁻²⁴. To investigate the molecular mechanisms of GA effects, the present inventors sought out to characterize the immune response of infarcted hearts after GA treatment. First, examining the changes in relevant immune populations. For that, infarcted hearts were analyzed by FACS using markers for neutrophils and macrophages. To follow the dynamics of the immune response after MI, injured hearts at 24 hr, 96 hr and 14 days post MI were harvested. As seen in FIGS. 5A-D, a trend was detected in these two immune cells populations: GA treatment decreased the fraction of neutrophils in the infarcted hearts 24 h post MI (FIGS. 5A, B), at the time point considered to be the peak of neutrophil infiltration into injured hearts. Of note, one of the PBS treated hearts did not show the typical expected neutrophil's infiltration at 24 h, thus resulting in the high standard deviation observed in FIG. 4B. The effect of GA diminishes after 96 hr, with the expected reduction in neutrophils. On the other hand, the macrophage population showed an opposite trend (FIGS. 5C, D). GA treated infarcted hearts showed an increase in the macrophage fraction of immune cells 24 hr post MI, which diminished after 96 hr. Taken together, this data reveals a GA-dependent antiparallel change in neutrophil and macrophage populations 24 hr post MI, wherein neutrophil numbers decrease while macrophage number increase. This trend was recently shown to promote cardiac regeneration²⁵, and therefore might explain GA regenerative effect.

### Example 6

### GA Alters Cytokine Levels in the Heart

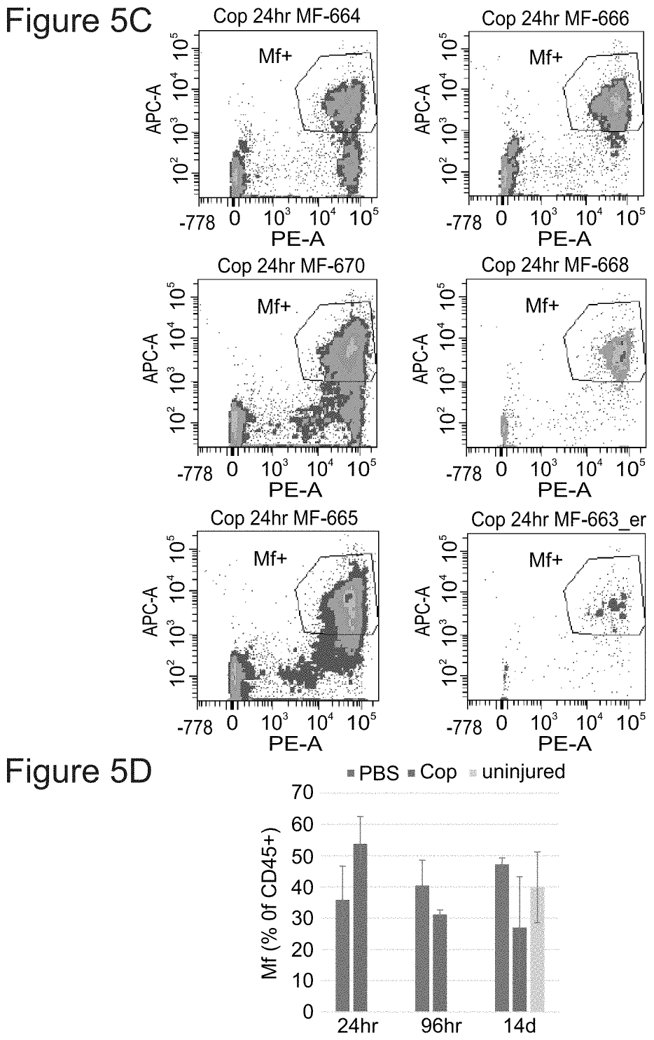

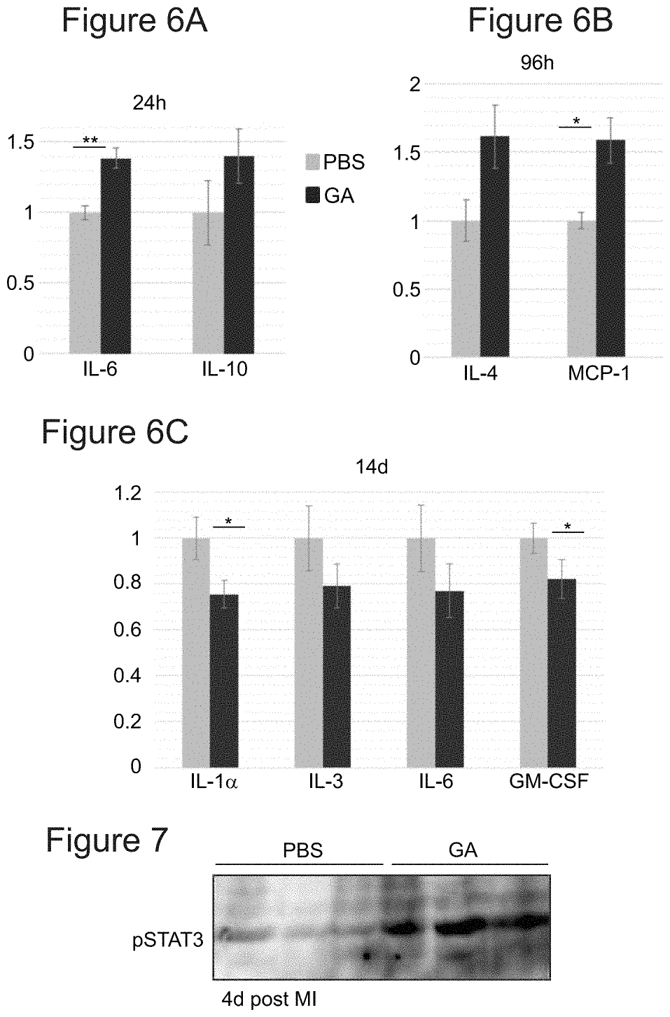

To broaden the understanding of the molecular effectors influenced by GA treatment, the cytokine milieu of the GA treated infarcted hearts was analyzed. For that, infarcted hearts were harvested at 24 h, 96 h and 14 days post MI. Native protein lysates were prepared and subjected to multiplex cytokine ELISA. At 24 hr post MI, both IL-6 and IL-10 levels were increased (FIG. 6A). IL-6 was previously shown to be up regulated by GA^{22, 24}, and was found to be required for neonatal cardiac regeneration^{26, 27} while its prolonged expression is deleterious and results in chronic heart failure²⁸. Importantly, the levels of IL-6 as well as those of other pro-inflammatory cytokines in GA treated hearts were reduced 14 days post MI (FIG. 6C). These findings might underscore the importance of regulating a transient expression of pro-inflammatory cytokines occurring in a pro-regenerative scenario, similar to what was detected in the GA treated hearts. Another cytokine that was elevated after GA treatment was IL-4 (FIG. 6B). Both IL-10 and IL-4 are considered as anti-inflammatory cytokines that induce Th-2 differentiation, a cell population previously shown to be affected by GA (40-42). GA treatment also induced a significant enhancement in the levels of Monocyte Chemoattractant Protein-1 (MCP-1), 96 hr post MI (FIG. 6B). This chemokine was previously shown to induce IL-6 expression and to have beneficial effect on cardiac remodeling post MI²⁹. Interestingly, the cytokines shown to be upregulated by GA; IL-10, IL-6 and MCP-1, can induce the activation of the transcription factor Stat3, shown to have beneficial effects repairing the heart after injury²⁹. Western-blot analysis of the activated phosphorylated form of Stat3 (pStat3), show its increased levels in GA treated hearts, 4 days after injury (FIG. 7). Taken together, the differential expression of cytokines in the GA treated infarcted heart might reveal the molecular mechanism that allows GA to modulate the immune response, supporting cardiac regeneration post MI.

### Example 7

### Beneficial Effects of Treatment with GA in Improving Heart Function Using a Chronic Heart Failure Model

Mice or rats are subjected to MI, and treatment with GA one-month post MI, to allow for a significant heart function deterioration and scar tissue formation before treatment. MI is evaluated 2 weeks post MI via Echo ultrasound cardiography, and animals without significant MI are excluded. GA or PBS is injected one-month post MI (i.p.). Heart function is evaluated by Echo 2 months post MI, and hearts will be explanted and subjected to basic histological analysis (Masson trichrome and H&E).

The experimental timeline is summarized below:

TABLE 1
Temporal description of chronic experiment

| Time point | | Monitor |
|---|---|---|
| (Days post MI) | Procedure | modalities |
| 0 | | Echo |
| | | (baseline) |
| 0 | LAD ligation | — |
| 14 | MI verification | Echo |
| 30 | GA/PBS administration | Echo |
| 60 | Sacrifice and pathology | Echo |

10 mice in each group are analyzed.

This study serves as a proof of concept for GA function in an injury setting. The mechanism in which GA improves heart function in CHF is also studied. GA treatment may be provided alone or in combination with other reparative cues.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### (Other References are Cited Throughout the Application)

1 organization Wh. World health statistics 2016. 2016
2 Sorita A, Ahmed A, Starr S R, Thompson K M, Reed D A, Prokop L, Shah N D, Murad M H, Ting H H. Off-hour presentation and outcomes in patients with acute myocardial infarction: Systematic review and meta-analysis. BMJ. 2014; 348:f7393
3 Toldo S, Abbate A. The nlrp3 inflammasome in acute myocardial infarction. Nat Rev Cardiol. 2017
4 Epelman S, Liu P P, Mann D L. Role of innate and adaptive immune mechanisms in cardiac injury and repair. Nat Rev Immunol. 2015:117-129; 15
5 Weirather J, Hofmann U D, Beyersdorf N, Ramos G C, Vogel B, Frey A, Ertl G, Kerkau T, Frantz S. Foxp3+ cd4+ t cells improve healing after myocardial infarction by modulating monocyte/macrophage differentiation. Circulation research. 2014; 115:55-67
6 Yan X, Anzai A, Katsumata Y, Matsuhashi T, Ito K, Endo J, Yamamoto T, Takeshima A, Shinmura K, Shen W, Fukuda K, Sano M. Temporal dynamics of cardiac immune cell accumulation following acute myocardial infarction. Journal of molecular and cellular cardiology. 2013; 62:24-35
7 Ziemssen T, Ashtamker N, Rubinchick S, Knappertz V, Comi G. Long-term safety and tolerability of glatiramer acetate 20 mg/ml in the treatment of relapsing forms of multiple sclerosis. Expert opinion on drug safety. 2017; 16247-255:
8 Aharoni R. The mechanism of action of glatiramer acetate in multiple sclerosis and beyond. Autoimmunity reviews. 2013; 12:543-553
9 Aharoni R. Immunomodulation neuroprotection and remyelination—the fundamental therapeutic effects of glatiramer acetate: A critical review. Journal of autoimmunity. 2014; 54:81-92
10 Weber M S, Prod'homme T, Youssef S, Dunn S E, Rundle C D, Lee L, Patarroyo J C, Stüve O, Sobel R A, Steinman L. Type ii monocytes modulate t cell-mediated central nervous system autoimmune disease. Nature medicine. 2007; 13:935
11 Aharoni R, Teitelbaum D, Sela M, Arnon R. Copolymer 1 induces t cells of the t helper type 2 that crossreact with myelin basic protein and suppress experimental autoimmune encephalomyelitis. Proceedings of the National Academy of Sciences. 1997; 94:10821-10826
12 Aharoni R, Teitelbaum D, Sela M, Arnon R. Bystander suppression of experimental autoimmune encephalomyelitis by t cell lines and clones of the th2 type induced by copolymer 1. Journal of neuroimmunology 91:135-146; 1998.
13 Neuhaus O, Farina C, Yassouridis A, Wiendl H, Bergh F T, Dose T, Wekerle H, Hohlfeld R. Multiple sclerosis: Comparison of copolymer-1-reactive t cell lines from treated and untreated subjects reveals cytokine shift from t helper 1 to t helper 2 cells. Proceedings of the National Academy of Sciences. 2000; 97:7452-7457
14 Duda P W, Schmied M C, Cook S L, Krieger J I, Hafler D A. Glatiramer acetate (Copaxone®) induces degenerate, th2-polarized immune responses in patients with multiple sclerosis. The Journal of clinical investigation. 2000; 105:967-976
15 Jee Y, Piao W H, Liu R, Bai X F, Rhodes S, Rodebaugh R, Campagnolo D I, Shi F D, Vollmer T L. Cd4+ cd25+ regulatory t cells contribute to the therapeutic effects of glatiramer acetate in experimental autoimmune encephalomyelitis. Clinical Immunology. 2007; 125:34-42
16 Aharoni R, Eilam R, Stock A, Vainshtein A, Shezen E, Gal H, Friedman N, Arnon R. Glatiramer acetate reduces th-17 inflammation and induces regulatory t-cells in the cns of mice with relapsing-remitting or chronic eae. Journal of neuroimmunology. 2010; 225:100-111
17 Aharoni R, Arnon R, Eilam R. Neurogenesis and neuroprotection induced by peripheral immunomodulatory treatment of experimental autoimmune encephalomyelitis. Journal of Neuroscience. 2005; 25:8217-8228
18 Aharoni R, Kayhan B, Arnon R. Therapeutic effect of the immunomodulator glatiramer acetate on trinitrobenzene sulfonic acid-induced experimental colitis. Inflammatory bowel diseases. 2005; 11:106-115
19 Aharoni R, Kayhan B, Brenner O, Domev H, Labunskay G, Arnon R. Immunomodulatory therapeutic effect of glatiramer acetate on several murine models of inflammatory bowel disease. Journal of Pharmacology and Experimental Therapeutics. 2006; 318:68-78
20 Aharoni R, Sonego H, Brenner O, Eilam R, Arnon R. The therapeutic effect of glatiramer acetate in a murine model of inflammatory bowel disease is mediated by anti-inflammatory t-cells. Immunology letters. 2007; 112:110-119
21 Pinto A R, Ilinykh A, Ivey M J, Kuwabara J T, D'Antoni M L, Debuque R, Chandran A, Wang L, Arora K, Rosenthal N A, Tallquist M D. Revisiting cardiac cellular composition. Circ Res. 2016; 118:400-409
22 Aharoni R, Teitelbaum D, Leitner O, Meshorer A, Sela M, Arnon R. Specific th2 cells accumulate in the central nervous system of mice protected against experimental autoimmune encephalomyelitis by copolymer 1. Proc Natl Acad Sci USA. 2000; 97:11472-11477
23 Aharoni R, Teitelbaum D, Sela M, Arnon R. Copolymer 1 induces t cells of the t helper type 2 that crossreact with myelin basic protein and suppress experimental autoimmune encephalomyelitis. Proc Natl Acad Sci USA. 1997; 94:10821-10826
24 Aharoni R, Teitelbaum D, Sela M, Arnon R. Bystander suppression of experimental autoimmune encephalomyelitis by t cell lines and clones of the th2 type induced by copolymer 1. J Neuroimmunol. 1998; 91:135-146
25 Lai S L, Marin-Juez R, Moura P L, Kuenne C, Lai J K H, Tsedeke A T, Guenther S, Looso M, Stainier D Y. Reciprocal analyses in zebrafish and medaka reveal that harnessing the immune response promotes cardiac regeneration. Elife. 2017; 6
26 Han C, Nie Y, Lian H, Liu R, He F, Huang H, Hu S. Acute inflammation stimulates a regenerative response in the neonatal mouse heart. Cell research. 2015; 25:1137-1151
27 Kai Sheng Q Z, Bingren Gao. Mechanisms of it-6 mediated early inflammation in cardiac regeneration of neonatal mice. Int J Clin Exp Med. 2018; 11:4530-4538
28 Fontes J A, Rose N R, Cihakova D. The varying faces of it-6: From cardiac protection to cardiac failure. Cytokine. 2015; 74:62-68
29 Morimoto H, Takahashi M, Izawa A, Ise H, Hongo M, Kolattukudy P E, Ikeda U. Cardiac overexpression of monocyte chemoattractant protein-1 in transgenic mice prevents cardiac dysfunction and remodeling after myocardial infarction. Circ Res. 2006; 99:891-899

What is claimed is:

1. A method of treating a heart disease in a subject in need thereof, the method comprising administering to the subject a therapeutically effective amount of an agent selected from the group consisting of Copolymer 1, a Copolymer 1-related polypeptide and a Copolymer 1-related peptide, thereby treating the heart disease, with the proviso that said agent is not administered to the subject in combination with stem cells, a diacylglycerol acyltransferase 2 (DGAT2) inhibitor or a ferroptosis inhibitor, and
when said heart disease is a result of an acute adverse cardiac event, administering commences up to 48 hours post said cardiac event for up to two weeks; or
when said heart disease is a chronic heart disease said disease is a chronic heart failure.

2. The method of claim 1, wherein said heart disease is an ischemic heart disease.

3. The method of claim 1, wherein said heart disease is a result of an acute adverse cardiac event.

4. The method of claim 1, wherein said heart disease is a result of a chronic stress or disease of the heart.

5. The method of claim 1, wherein said therapeutically effective amount results in a functional and/or anatomical repair of a damaged heart tissue.

6. The method of claim 1, wherein said functional repair comprises an increase in cardiac output.

7. The method of claim 1, wherein said increase in cardiac output comprises an increase in left ventricular ejection fraction (LVEF) of at least 5%.

8. The method of claim 1, wherein said increase in cardiac output comprises an increase in left ventricular fractional shortening (LVFS) of at least 2%.

9. The method of claim 1, wherein said therapeutically effective amount results in an anatomical repair of a damaged or diseased tissue comprising an increase in ventricular wall thickness and/or a decrease in scar tissue formation.

10. The method of claim 1, wherein said therapeutically effective amount results in a modification in expression of cytokines at a cardiac lesion site.

11. The method of claim 1, wherein said therapeutically effective amount results in Stat3 activation.

12. The method of claim 1, wherein said therapeutically effective amount results neutrophils decrease and macrophages increase at a cardiac lesion site.

13. The method of claim 1, wherein said agent is Copolymer 1.

14. The method of claim 1, wherein the subject does not suffer from multiple sclerosis.
